(19) United States
(12) Patent Application Publication
(10) Pub. No.: US 2011/0183422 A1
(43) Pub. Date: Jul. 28, 2011

(54) MEASUREMENT OF CURING

(75) Inventors: Leif Cronvall, Kivik (SE); Jonas Lundberg, Torna-Hallestad (SE)

(73) Assignee: REOSENSE AB, Lund (SE)

(21) Appl. No.: 13/003,219

(22) PCT Filed: Jun. 29, 2009
(86) PCT No.: PCT/SE09/50830

§ 371 Date: Apr. 12, 2011

Related U.S. Application Data
(60) Provisional application No. 61/129,569, filed on Jul. 7, 2008.
(60) Provisional application No. 61/193,348, filed on Nov. 20, 2008.

(51) Int. Cl. G01N 33/44

(52) U.S. Cl. 436/34

(57) ABSTRACT

A method for increasing productivity and quality in production of cured polymer by a real time measurement of the progress of curing, in a mould cavity, which being filled with a compound to be cured, wherein a signal is generated by a wave at an ultrasonic or equivalent frequency, which is transmitted through the mould cavity, the time for the wave to pass through the compound in the mould and back is detected, and the detected signal being analyzed in a suitable data processing computer , to establish a graph showing a relationship between the time for the wave to pass through the compound in the mould and back, and the time of the curing of the compound. The graph is used to determine a feature of the compound by identifying at least one specific parameter of the graph, and using said parameter to control the production.

### TECHNICAL FIELD

The present invention refers to a method for increasing productivity and quality in production of cured polymer, such as vulcanization of rubber or thermosetting of plastic, by a real time measurement of the progress of curing, in a mould cavity, which being filled with a compound to be cured, wherein a signal is generated by a wave at an ultrasonic or equivalent frequency, which is transmitted through the mould cavity, the time for the wave to pass through the compound in the mould and back is detected, and the detected signal being analyzed in a suitable data processing computer, to establish a graph showing a relationship between the time for the wave to pass through the compound in the mould and back, and the time of the curing of the compound.

### BACKGROUND OF THE INVENTION

A method for monitoring a vulcanization process of introductory described kind is previously known by DE 101 38 791, in which the vulcanization mixture being treated with ultrasound during vulcanization. The detected signal is used to establish a graph showing a relationship between the time for the wave to pass through the mixture in the mould and back and the time of the curing of the mixture. However, this reference does not disclose any practical use of the information.

A measurement of the degree of progress in vulcanization of rubber is previously known by JP1110255, which shows an ultrasonic vibrator and an ultrasonic sensor to detect the amplitude of an ultrasonic wave through a rubber sample. The detected value from the sensor is compared with a reference value and being used to control the supply power to the ultrasonic vibrator. From this reference it is mentioned that the damping force of the rubber sample relative to the ultrasonic wave is in close relation with the degree of proceeding in the vulcanization of the rubber. Thus, the ultrasonic vibrator is used to influence the vulcanization of the rubber. However, the reference refers to a rubber sample and does not disclose any measuring of the rubber curing on-line in a production mould. Due to the variations in the properties of the rubber compounds, the curing velocity for the same recipe can differ as much as 30%, but the operator of the moulding machine normally uses the same machine time for all batches, often with an extra machine time built into the cycle time to be sure that all the products are cured properly.

Purpose of the invention

With the present invention a method is disclosed, in which a realtime ultrasonic measurements in injection mould shows the time of maximal number of cross-links. An algorithm will make it possible to have an automatic opening of the press. An issue is that when shortening the cure time, the values of compression set increase.

With the present invention a method is disclosed, which monitors the rubber curing on-line in the mould and the machine time is adjusted automatically to the real curing time, which gives several benefits. The cycle time will be reduced which increases the production capacity approximately by 10-30%. Furthermore, a more stable product quality can be obtained with tighter tolerances for properties such as hardness, ultimate strength, rigidity and compression set. The invention may be used to control the press to open after a specific progress of the curing, when the product is ready, instead of opening after a fixed time, which is typical today.

### SUMMARY OF THE INVENTION

The purpose with the present invention is to provide a method wherein the graph is used to determine a feature of the compound by identifying at least one specific parameter of the graph, and using said parameter to control the production. The graph may be used to determine a maximum number of cross-links in the compound, or to determine when the compound has settled in the mould.

Suitable a gradient of the graph is identified as a specific parameter. In addition, a derivative value of the graph is identified as a specific parameter. Preferably, a maximum derivative value is identified.

In a preferred embodiment, the time of a maximum derivative value being used as a triggering point, to a desired cure time.

In a second embodiment, the time of an increasing derivative value plus a preset factor being used as a triggering point, to a desired cure time.

The purpose with the present invention is to provide a method wherein the graph is used to determine when the compound has settle in the mould cavity by identifying at least one specific parameter of the graph, and using said parameter to control the production.

Suitable specific parameter to be identified includes a starting level of the graph, a gradient of the graph and a break point value of the graph.

The break point value corresponds to a point where the gradient essentially has flattened out and preferably the break point value corresponds to a peak of the graph, where the pitch angle of the gradient is zero.

An advantage is that the break point value is used to calculate a sufficient curing time and that the curing time is used to control the mould to open.

The present invention also refers to a means for increasing productivity and quality in production of cured polymer, such as vulcanization of rubber or thermosetting of plastic, by detecting the progress of curing, comprising a mould for production of cured polymer having a cavity filled with a compound to be cured, a transmitter for generating a signal in shape of a wave at an ultrasonic or equivalent frequency, and a receiver for the signal which being transmitted through the mould cavity, and an apparatus to measure the time for the wave to pass through the compound in the mould and back in real time measurement of the signal, and a suitable data processing computer for analyzing the detected signal, the detected signal is transformed into a graph showing a relationship between the time for the wave to pass through the compound in the mould and back, defined as time of flight, and the time of the curing of the compound, comprising a detection device for identifying at least one specific parameter of the graph, and using said parameter to control the production.

Preferably the means comprising a device adding the real time value of said parameter to a desired time value to calculate a sufficient curing time, and a control means for opening the mould in view of the curing time.

Other objectives, features and advantages of the present invention will appear from the following detailed disclosure, from the attached claims as well as from the drawings.

Generally, all terms used in the claims are to be interpreted according to their ordinary meaning in the technical field, unless explicitly defined otherwise herein.

### BRIEF DESCRIPTION OF THE DRAWINGS

The above, as well as additional objects, features and advantages of the present invention, will be better understood through the following illustrative and non-limiting detailed description of preferred embodiments of the present invention, with reference to the appended drawings, where the same reference numerals will be used for similar elements, wherein:

FIG. 1 is a schematic view of a general injection moulding apparatus to perform the method according to the present invention,

FIGS. 2a and 2b are graphs showing a relationship between the time of flight of an ultrasonic wave in different rubber compounds and the time of the curing of the rubber compounds,

FIG. 3 is a graph showing a relationship between the temperature in a rubber compound and the time of the curing of the rubber compound,

FIG. 4 is a graph showing a relationship between the pressure in a rubber compound and the time of the curing of the rubber compound,

FIG. 5 is a graph showing a relationship between the derivative related to pressure and temperature in a rubber compound and the time of the curing of the rubber compound, and a graph showing a relationship between the derivative of the time of flight of an ultrasonic wave in a rubber compound and the time of the curing of the rubber compound,

FIGS. 6 and 7 are different graphs showing the time of flight derivative and the spring rate of batches having different characteristics,

FIG. 8 is a graph showing the influence of a trigger level to the cure time,

FIG. 9 is a diagram showing variation in spring rate between an automatic cure time according to the present invention and a traditional fixed cure time,

FIG. 10 is a graph showing a relationship between the pressure and the temperature in a rubber compound having high sulphur content and the time of the curing of the rubber compound,

FIG. 11 are graphs showing a relationship between the time of flight and the derivative of the time of flight of an ultrasonic wave in a rubber compound and the time of the curing of the rubber compound,

FIG. 12 is a graph showing a relationship between the derivative of the time of flight of an ultrasonic wave in rubber compounds having different sulphur content and the time of the curing of the rubber compounds.

### DETAILED DESCRIPTION OF PREFERRED EMBODIMENTS

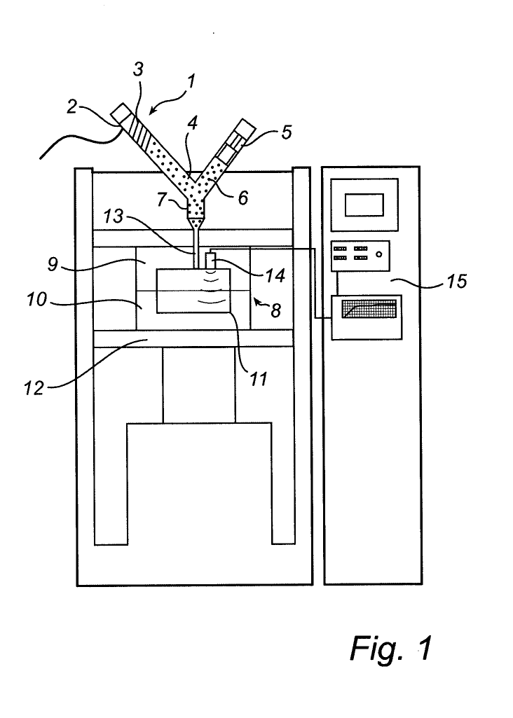

FIG. 1a illustrates a general injection moulding apparatus 1 having an inlet 2 with a heater 3 for a rubber or polymer compound. Adjacent to the outlet 4 of the heater is a hydraulic cylinder 5 located, which being filled with the heated compound 6. When a sufficient amount has been collected in the hydraulic cylinder 5 an injection nozzle 7 is opened and by aid of the pressure from the hydraulic cylinder 5 the compound 6 is injected through the injection nozzle 7 and into a mould 8 of suitable shape.

Preferably, a two-part or multi-part mould 8 is provided comprising a top plate 9 and a bottom plate 10, which when brought together define one or more mould cavities 11. The mould plates 9, 10 may, in a known manner, be provided with heating devices (not shown) for a preheating to about 170° C.

A conventional press table 12 presses the mould plates 9, 10 against each other. The injection nozzle 7 is received in a bore 13 in the top mould plate 9 and is retained therein. The bore 13 opens into one or more channels or passages (not shown) formed in the mould plates 9, 10 and constituting a communication between the hydraulic cylinder 5 and the mould cavity 11.

A transmitter and a sensor 14, either separated or combined, are attached to or built into the mould 8 close to the mould cavity 11 for transmitting and receiving a signal, which is generated by a wave at an ultrasonic or equivalent frequency, through the mould cavity 11, which being filled with the compound 6 to be cured. The mould 8 is also provided with a temperature sensor and a pressure sensor (not shown). Preferably, a wave-guide with low acoustic impedance has been mounted inside an injection mould 8, in contact with the cavity 11. An ultrasonic transducer has been mounted on the outside, in contact with the wave-guide. The transducer has transmitted and received an ultrasonic pulse that has propagated through the rubber and back.

Means for analyzing the detected signal in a suitable data processing computer 15 is provided and comprising an apparatus (not shown) to measure the time for the wave to pass through the compound in the mould and back in real time measurement of the signal, and a signal processor (not shown) wherein the detected signal is transformed into a graph showing a relationship between the time for the wave to pass through the compound in the mould and back, defined as time of flight, and the time of the curing of the compound, and a detection device (not shown) for identifying a break point value of the graph. Preferably, said means comprising a device (not shown) adding the real time value of the break point value to a desired time value to calculate a sufficient curing time, and a control means (not shown) for opening the mould in view of the curing time.

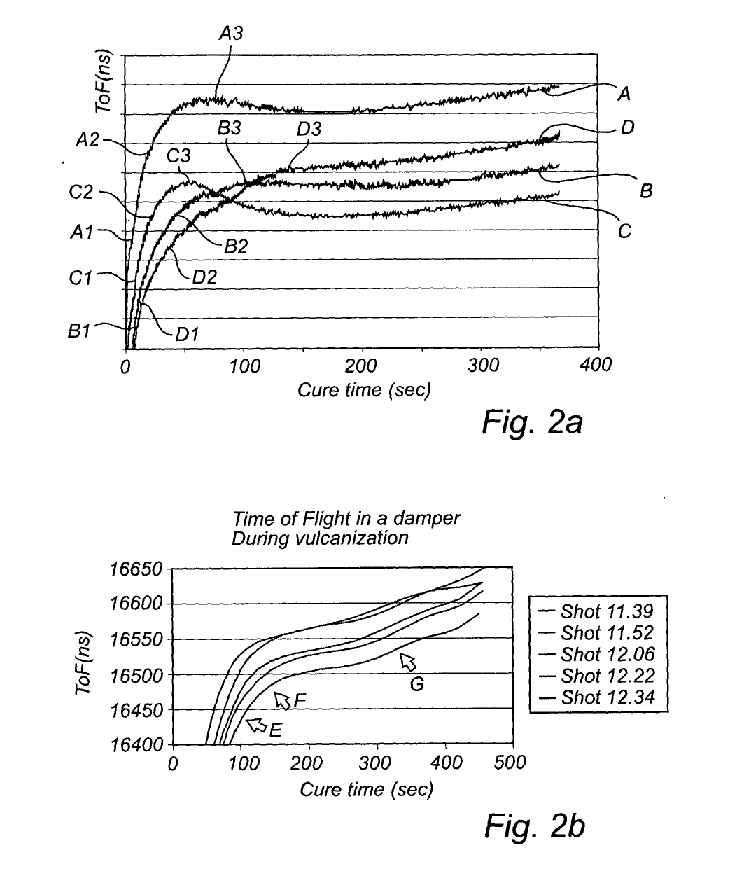

FIG. 2a is a graph showing a relationship between the time of an ultrasonic wave to pass through the compound in the mould 8 and back, defined as time of flight, and the time of the curing of the compound. By detecting the time of flight for the wave, and analyzing the detected signal in a suitable data processing computer 15 it is possible to determinate when the compound has settle in the mould cavity 11 and the progress of the curing, by identifying at least one specific parameter of the graph, and using said parameter to control the production, e.g. such that the data being used to controls the press 12 to open when the curing is ready, instead of opening after a fixed time, which is typical today.

The graph, as shown in FIG. 2a, disclose the time for the progress of the curing of the compound, expressed in seconds, plotted against the time of flight of the ultrasonic wave, which is measured by suitable receiver sensors. It should be noted for the sake of clear understanding of the present invention that while the ultrasonic wave measuring device is known in the art, and therefore need not be described here in detail, the use of the measured ultrasonic wave for determining time of curing is new, and constitutes an important aspect of the present invention.

In FIG. 2a the graph of three different types of rubber compounds are shown. The first graph A represents a normal rubber compound with a high injection velocity. The second graph B represents a normal rubber compound with normal injection velocity. The third graph C represents a fast rubber compound with normal injection velocity. The fourth graph D represents a normal rubber compound injected at lower temperature. The properties of the rubber compounds are dependant of several factors, e.g. the recipe of the compound, the pre-vulcanization of the compound, due to aging, but also the temperature of the rubber, the temperature of the mould, and also the injection velocity to the mould and the viscosity of the compound. Due to the variations in the properties of the rubber compounds and in the process, the curing velocity for the same recipe can differ as much as 30%, but the operator of the moulding machine of hitherto known kind normally uses the same machine time for all batches, often with an extra machine time built into the cycle time to be sure that all the products are cured properly. With the present invention the machine time is adjusted to the real curing time, which gives several benefits. The cycle time will be reduced, which increases the production capacity of about 10% to 30%. Furthermore, a more stable product quality can be obtained with tighter tolerances for properties such as hardness, ultimate strength, rigidity and compression set.

The time of flight increases with increased temperature and decreases with increased pressure. As is evident from the graphs A, B, C, D shown in FIG. 2a it is possible to identifying specific parameters of the graph by measurement of the time of flight to establish a starting level A₁, B₁, C₁, D₁ of each graph, which depends on the starting temperature of the compound, and it is also possible to detect the gradient A₂, B₂, C₂, D₂ of each graph, which depends on the temperature of the mould. Consequently, a lower temperature prolongs the time for curing and a higher temperature reduces the time for curing. Finally, it is possible to establish a break point value A₃, B₃, C₃, D₃ of each graph, corresponding to the peak of the graph, where the pitch angle of the graph is zero, or to a point where the gradient essentially has flatten out, which depends of the properties of the compound, as well as of the temperature of the mould, and corresponds to a curing of the compound wherein the compound has settle in the mould cavity, i.e. when the cross-linking of the compound has reached a level when it will change phase and solidify in the mould. The settlement is believed to occur when the curing compound seals towards the bore 13 of the injection nozzle 7 and towards any gap between the plates 9, 10 of the mould 8, resulting in an increased pressure in the mould, which consequently reduces the time of flight and corresponds to said break point value.

According to the present invention the break point value A₃, B₃, C₃, D₃ is determined by a real time measurement of the progress of the curing process and it has now become evident that the difference in curing time between different batches corresponds to the time it takes to reach the break point value as appear in the time of flight measurement. By this knowledge it is possible to calculate a sufficient curing time for each batch or adjust the curing time if the value deviates from a desired value.

By the present invention it is thus possible to identify a suitable graph, or an interval between a desired upper limit graph and a lower limit graph, for a specific product produced by e.g. an injection moulding process, which corresponds to a desired quality of the product. If the real time measurement shows a graph that differs from the desired interval it is possible to shorten or extend the curing time. In FIG. 8 a trigger level set shows that a rubber compound with fast curing properties having a shorter cure time than a rubber compound with slow curing properties, which is possible to enable with a method according to the present invention. However, it is also possible to adjust to the desired interval by changing the starting temperature or by changing the properties of the compound, or by changing the injection velocity or the temperature of the mould.

It is also possible to combine the measurement of the time for the wave to pass through the compound in the mould and back with a real time measurement of the pressure by aid of said pressure sensor. By doing that and compensate the detected signal from the wave with the input from the pressure sensor it is possible to calculate the temperature within the mould.

FIG. 2b show that the ToF responds to the vulcanization. A change in the relation between temperature and pressure has a significant impact on the ToF. During the cure time, the temperature is normally increasing. As shown by FIG. 4 the pressure will fall in the beginning due to that the rubber heats up and spread. Because of this, as shown at E in FIG. 2b, the ToF will increase considerably in the beginning.

At a point, when vulcanization starts in the area close to the cavity wall, the floating will decrease and the pressure drop will slow down and the pressure will start to increase instead. As shown at F in FIG. 2b this will reduce the rise of ToF.

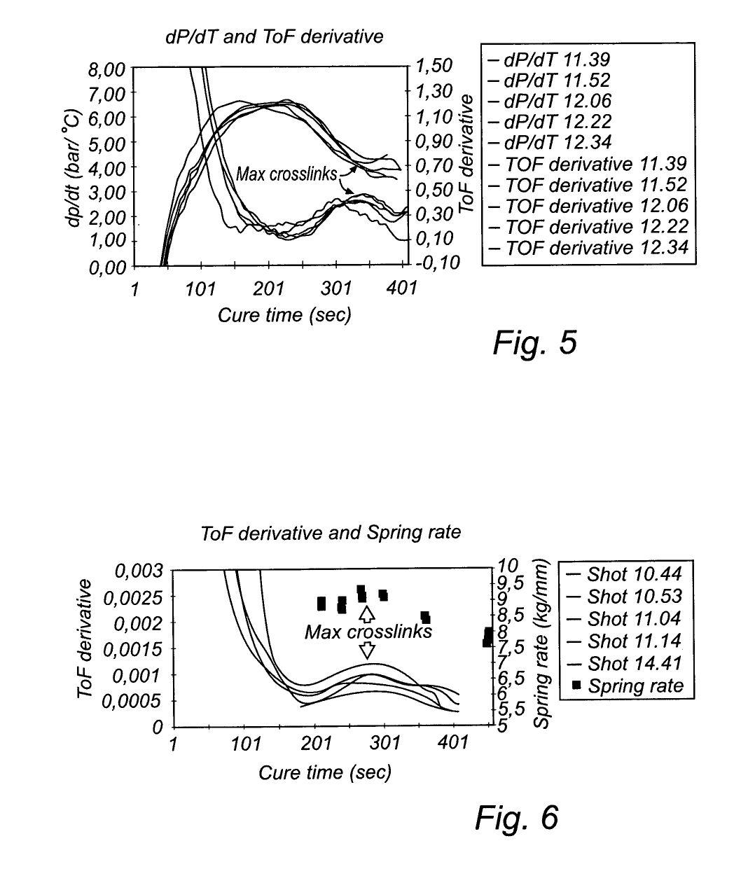

As shown at G in FIG. 2b the ToF is increasing again, which is a result of a change in the relation between pressure and temperature. By using the detected signals it is also possible to calculate a graph as function of a derivative dP/dT related to pressure and temperature vs. the time of curing, as being showed in FIG. 5. From this graph it will be evident that the graph will reach a peak value when the compound is settled in the mould and thereafter the graph will stabilizing at an essential constant level, which support a direct proportional between pressure and temperature.

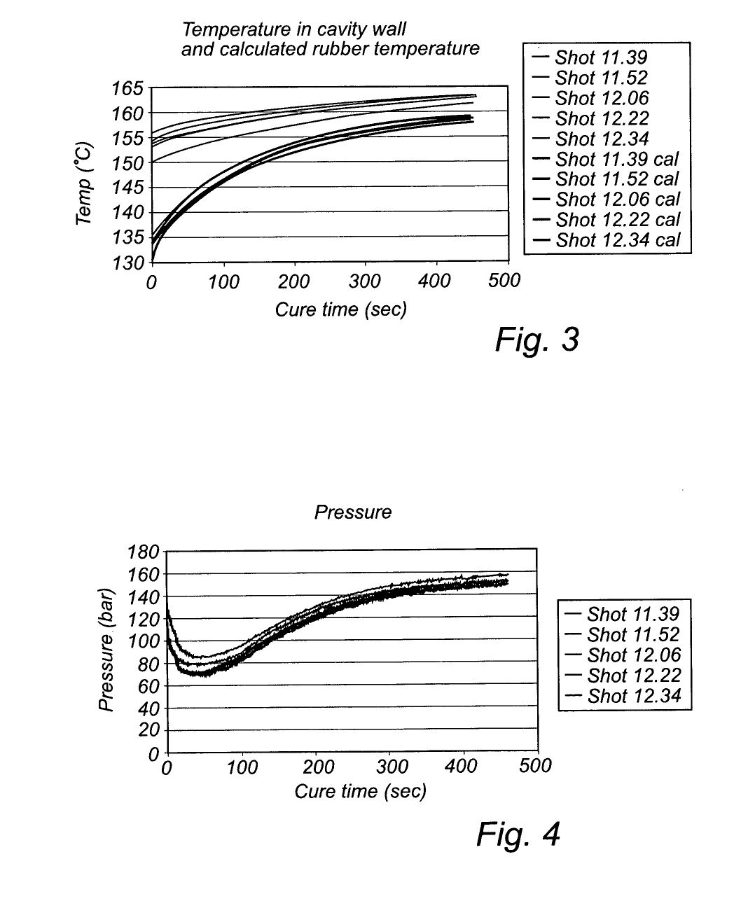

In FIGS. 3 and 4 both temperature and pressure have been measured in addition to ultrasound during the tests.

FIG. 5 illustrate the derivative dP/dT related to pressure and temperature, which indicates how much the pressure increases for each degree increase in temperature. FIG. 5 shows there is a maximum value at 150-200 seconds of 6,5 bar per C° . This is the time when the mould is sealed. At the time of approx. 350 seconds, dP/dT has decreased and levels out to 4 bar per C° . This is due to the cross-links that have a shrink effect on the rubber. The forces of the cross-links counteract on the expansion forces. Therefore, the coefficient of thermal expansion changes at this point. Support for these theories can be found from experts like B Stenberg and S Persson.

FIG. 5 shows also the derivative of ToF and the maximum derivatives have been pointed out as “Max crosslinks”. The maximum amount of cross-links appears when dP/dT has reached its lowest value, which corresponds with when the ToF has the most positive derivative, see G in FIG. 2b.

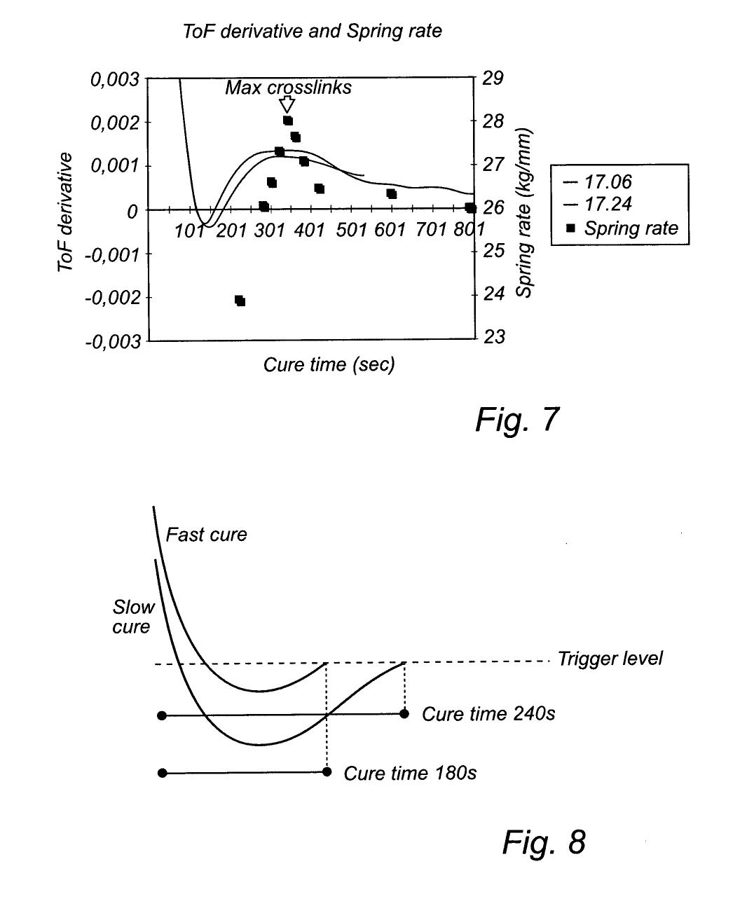

Referring to FIGS. 6 and 7 a series of shots with decreasing cure time has been performed, which strengthen the statement that the time for maximum derivative is the time of maximal number of cross-links. In addition, the spring rate for each shot has been measured.

An issue is that by shortening the cure time, the values for compression set are increasing. The ToF curve is used to decide a triggering point for the algorithm to a desired cure time. The result could be that for some compounds, it will be the time of the maximum derivative and for other compounds, it will be the time when the derivative turns upwards plus a preset factor.

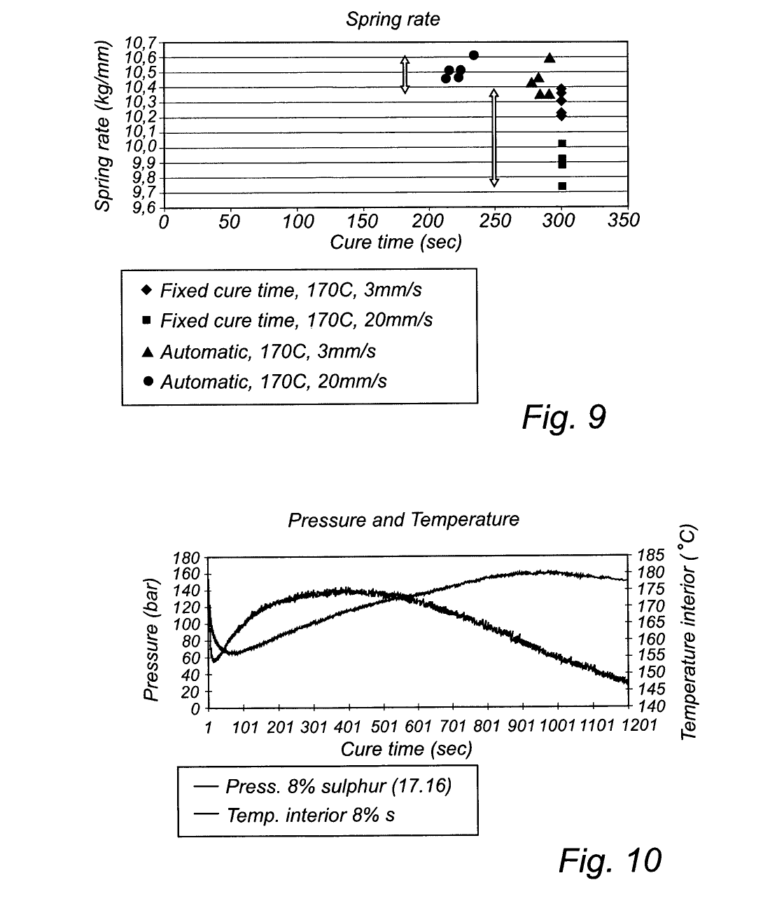

By the present invention, a way to measure the effects of cross-links in real time has been found, and the ToF signal responds to the amount of cross-links. An algorithm will be based on different trigger points, and the algorithm will adjusting for variations in mould temperature and compound, with the result of a more consistent quality and a shorter cure time. In FIG. 9 the variation in spring rate between an automatic cure time according to the present invention and a traditional fixed cure time is shown and the result is that the automatic cure time provides less variation and consequently a higher quality.

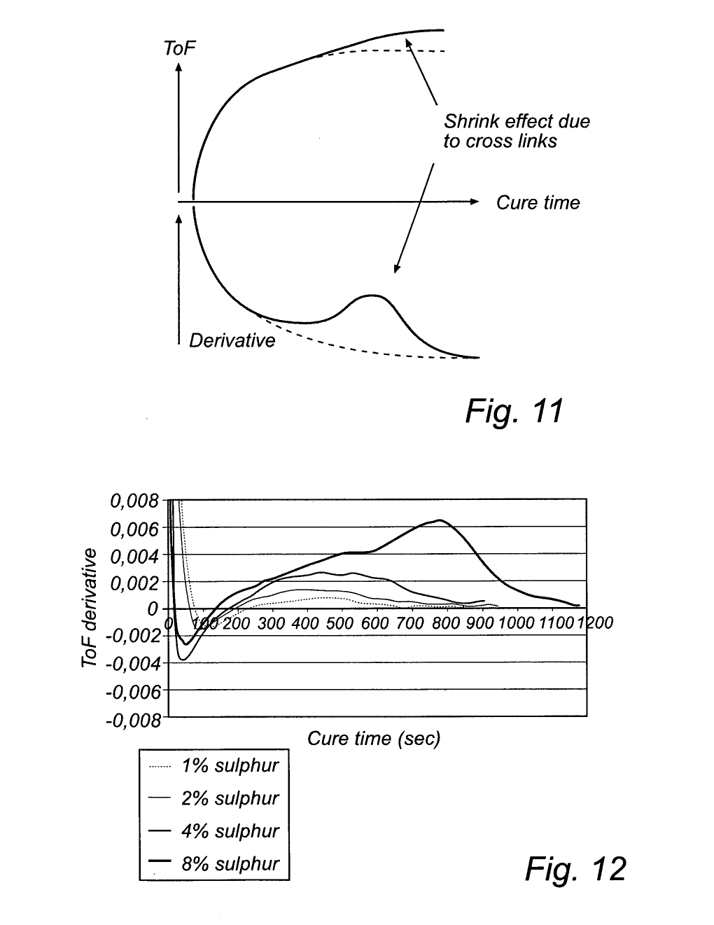

In FIG. 10 a rubber compound having high sulphur content, which contributes to the cross-links in the rubber compound, is measured during curing. As evident from the graph, the pressure decrease despite increasing temperature, which indicates a non-linear thermal expansion of the rubber and a shrink effect during curing. Such shrink effect due to formation of cross-links in the rubber compound may be detected, as shown in FIG. 11, by the method according to the present invention.

The significance of different sulphur content of the rubber compounds is shown in FIG. 12 and verifies the shrinking effect.

The invention has mainly been described above with reference to a few embodiments. However, as is readily appreciated by a person skilled in the art, other embodiments than the ones disclosed above are equally possible within the scope of the invention, as defined by the appended patent claims, e.g. the ultrasonic measurement could be combined with other methods of measurement, including microwaves.

It will be understood that the invention is not restricted to the example described and that the mould may be of any conventional type and may be filled with the compound by other well-known means, instead of by injection moulding.

1. A method for increasing productivity and quality in production of cured polymer, by a real time measurement of the progress of curing, in a mould cavity, which being filled with a compound to be cured, wherein a signal is generated by a wave at an ultrasonic or equivalent frequency, which is transmitted through the mould cavity, the time for the wave to pass through the compound in the mould and back is detected, and the detected signal being analyzed in a suitable data processing computer, to establish a graph showing a relationship between the time for the wave to pass through the compound in the mould and back, and the time of the curing of the compound, wherein the graph is used to determine a feature of the compound by identifying at least one specific parameter of the graph, and using said parameter to control the production.

2. A method according to claim 1, wherein the graph is used to determine a maximum number of cross-links in the compound.

3. A method according to claim 2, wherein a gradient of the graph is identified as a specific parameter.

4. A method according to claim 2, wherein a derivative value of the graph is identified as a specific parameter.

5. A method according to claim 4, wherein the time of a maximum derivative value being used as a triggering point to a desired cure time.

6. A method according to claim 4, wherein the time of an increasing derivative value plus a preset factor being used as a triggering point to a desired cure time.

7. A method according to claim 1, wherein the graph is used to determine when the compound has settled in the mould.

8. A method according to claim 7, wherein a starting level of the graph is identified as a specific parameter.

9. A method according to claim 7, wherein a gradient of the graph is identified as a specific parameter.

10. A method according to claim 9, wherein a break point value of the graph is identified as a specific parameter.

11. A method according to claim 10, wherein the break point value corresponds to a point where the gradient essentially has flattens out.

12. A method according to claim 11, wherein the break point value corresponds to a peak of the graph, where the pitch angle of the gradient is zero.

13. A method according to claims 12, wherein the break point value is used to calculate a sufficient curing time.

14. A method according to claim 13, wherein the curing time is used to control the mould to open.

15. A method according to claim 7, wherein a combination of more than one parameter of the graph being used to control the production.

16. A method according to claim 7, wherein an interval between a desired upper limit graph and a lower limit graph is identified for a specific product, and as a result of the real time measurement adjusting the temperature of the compound to establish a starting level for the graph of said compound, which is within said interval.

17. A method according to claims 9, wherein an interval between a desired upper limit graph and a lower limit graph is identified for a specific product, and as a result of the real time measurement adjusting the velocity of filling the mould to establish a gradient for the graph of said compound, which is within said interval.

18. A means for increasing productivity and quality in production of cured polymer, by detecting the progress of curing, comprising a mould for production of cured polymer having a cavity filled with a compound to be cured, a transmitter for generating a signal in shape of a wave at an ultrasonic or equivalent frequency, and a receiver for the signal which being transmitted through the mould cavity, and an apparatus to measure the time for the wave to pass through the compound in the mould if and back in real time measurement of the signal, and a suitable data processing computer for analyzing the detected signal, the detected signal is transformed into a graph showing a relationship between the time for the wave to pass through the compound in the mould and back, defined as time of flight, and the time of the curing of the compound, comprising a detection device for identifying at least one specific parameter of the graph, and using said parameter to control the production.

19. A means according to claim 18, comprising a device adding the real time value of said parameter to a desired time value to calculate a sufficient curing time.

20. A means according to claim 19, comprising a control means for opening the mould in view of the curing time.

21. The method of claim 1, wherein the production of cured polymer is a vulcanization of rubber or thermosetting of plastic.
